(19) United States
(12) Patent Application Publication
(10) Pub. No.: US 2024/0157053 A1
(43) Pub. Date: May 16, 2024

(54) USE OF NON-INVASIVE GLUCOSE SENSORS AND GLUCOSE RATE OF CHANGE DATA IN AN INSULIN DELIVERY SYSTEM

(71) Applicant: INSULET CORPORATION, Acton, MA (US)

(72) Inventors: Joon Bok LEE, Acton, MA (US); John D’ARCO, Wilmington, MA (US); Yibin ZHENG, Hartland, WI (US); Jason O’CONNOR, Acton, MA (US); James CAUSEY, Simi Valley, CA (US); Ashutosh ZADE, San Diego, CA (US)

(21) Appl. No.: 18/498,275

(22) Filed: Oct. 31, 2023

Related U.S. Application Data
(60) Provisional application No. 63/382,152, filed on Nov. 3, 2022.

(51) Int. Cl. A61M 5/172; A61M 5/142; G16H 20/17

(52) U.S. Cl. CPC A61M 5/1723; A61M 5/14244; G16H 20/17; A61M 2005/1401; A61M 2005/1726

(57) ABSTRACT

Invasive glucose sensors and noninvasive glucose sensors may be used in conjunction to improve glucose management for a user. The rate of change (ROC) of glucose levels from a noninvasive glucose sensor may be used rather than or in conjunction with a glucose level of the user from a CGM. A basal insulin delivery rate to the user may be adjusted responsive to the ROC glucose level data from the noninvasive sensor. The glucose level ROC from a noninvasive glucose sensor may be used to predict future glucose level ROCs of the user between operational cycles of an insulin delivery device and/or to identify possible hypoglycemic or hyperglycemic events. These predicted future glucose level ROCs may be used in a cost function of the control system of the insulin delivery device to select basal insulin delivery doses. Glucose level readings may be used to calibrate a noninvasive glucose level sensor.

### RELATED APPLICATIONS

This application claims the benefit of U.S. Provisional Patent Application No. 63/382,152, filed Nov. 3, 2022, the entire contents of which are incorporated herein by reference in its entirety.

### BACKGROUND

Glucose sensors may be invasive or noninvasive. An example of an invasive glucose sensor is a continuous glucose monitor (CGM). A CGM is secured to a user and includes a sensing element that is positioned subcutaneously in an interstitial space in the body of the user. The sensing element is able to sense data for determining a glucose level of the user. Typically, the CGM outputs a glucose level reading of the user at regular intervals, such as every five minutes or upon demand, to an insulin delivery device or a management device for the insulin delivery device. The management device may forward the glucose level reading to the insulin delivery device. The insulin delivery device may use the glucose level reading to determine a basal insulin dose to be delivered to the user.

Noninvasive glucose sensors include technologies that use electrochemical, spectroscopy, and/or light sensing. Noninvasive electrochemical sensors may measure body fluids, such as tears or sweat. An example is Integrity's GlucoTrack. Spectroscopic sensors use electromagnetic energy in an antenna array that excites whole blood glucose while measuring the magnitude of the excitement and the phase shift. Examples of such spectroscopic sensors are Hagar Tech's GWave. A form of light based sensors use laser sources and observe the reflectance of the output light to determine absorbance and glucose level rate of change (ROC) for the user. An example of such a noninvasive glucose sensor is the Rockley Bioptx wristband. Some solutions may use a combination of these technologies in addition to other biometric sensing like body temperature, oxygen saturation, heart rate, heart rate variability, respiration rate and blood pressure.

### SUMMARY

In accordance with an inventive facet, an insulin delivery system for delivering insulin to a user includes a non-transitory computer-readable storage medium storing processor-executable instructions. The insulin delivery device also includes a processor for executing the processor-executable instructions. Execution of the processor-executable instructions causes the processor to receive rate of change (ROC) data regarding a ROC of a glucose level of the user. In addition, execution of the processor-executable instructions causes the processor to use the ROC data to determine basal insulin delivery dosages and may also determine, in a cost assessment in a model predictive control algorithm, for example, a lowest glucose cost among candidate basal or microbolus insulin delivery dosages. Execution of the processor-executable instructions may cause the processor to use the ROC data to confirm that insulin was delivered to the user. Each of these may be determined using only the ROC data and not glucose level data itself.

As suggested above, the processor may use the ROC data without using glucose level data to determine basal insulin delivery dosages. The processor-executable instructions may cause the processor to analyze the ROC data to identify that a projected glucose level increase is projected. The processor-executable instructions may cause the processor to increase an insulin delivery rate by the insulin delivery device to the user to compensate for the projected glucose level increase. The user may be notified of a potentially imminent hypoglycemic event as well. The ROC change data may be available more quickly to identify such a potentially imminent hypoglycemic event than with a conventional glucose monitor, like a CGM. A magnitude of the increase of the insulin delivery rate may depend upon the ROC data and a target glucose level of the user. The processor may use the ROC data without using glucose level data to determine basal insulin delivery dosages. The processor-executable instructions may cause the processor to analyze the ROC data and determine that a decrease in glucose level is projected.

The processor-executable instructions may further cause the processor to decrease an insulin delivery rate or suspend insulin delivery by the insulin delivery device to the user to compensate for the projected glucose level decrease, e.g. decrease the insulin delivery rate or suspend insulin delivery such that the projected glucose level lies within a target range or deviates a minimum from the target range. The processor may use the ROC data without using the glucose level data to determine glucose cost of candidate basal insulin delivery dosages, and the processor-executable instructions may cause the processor to predict an ROC for a time period from the rates of change of preceding time periods. The processor-executable instructions may further cause the processor to use a cost function that predicts costs of candidate insulin doses using the predicted ROC for the time period. The cost function may include a glucose cost component that is determined based on the predicted ROC for the time period. The processor-executable instructions may further cause the processor to choose a selected one of the candidate insulin doses with a lowest cost as determined by the cost function and to cause the selected one of the insulin doses to be delivered to the user.

In accordance with another inventive facet, an insulin delivery system for delivering insulin to a user includes a non-transitory computer-readable storage medium storing processor-executable instructions and a processor for executing the processor-executable instructions. The instructions cause the processor to receive ROC data from a noninvasive sensor. The ROC data indicates an ROC of glucose concentration for the user. The instructions further cause the processor to modify a weight coefficient of a glucose cost component of a cost function based on the ROC data and to select an insulin dose among candidate insulin doses to be delivered to the user in an operational cycle of the drug delivery device using the cost function. The selected dose has a better cost relative to others of the candidate insulin doses. The instructions also cause the processor to cause the selected insulin dose to be delivered to the user during the operational cycle. The term “better” cost may relate to maximum or minimum value of the cost function, depending on the cost function. For example, a cost function wherein a deviation of the blood glucose values from a target value and a deviation of the insulin delivery from a baseline insulin delivery result in an increased cost, a “better” cost may be the minimum of said cost function.

The cost function may include a glucose cost component and an insulin cost component. The glucose cost component may be based on how much predicted glucose concentrations of the user will vary from a target value if a given insulin dose is delivered to the user in a current operational cycle of the drug delivery device. A weight coefficient of the glucose cost component may be calculated using the ROC data. The weight coefficient of the glucose cost component may increase a magnitude of the glucose cost component if the ROC data indicates that the glucose concentration is increasing and if a difference between the glucose concentration of the user for the current operational cycle and a glucose concentration of the user for a predecessor operational cycle is positive, or if the glucose concentration is decreasing and if a difference between the glucose concentration of the user for the current operational cycle and a glucose concentration of the user for a predecessor operational cycle is negative. The weight coefficient of the glucose cost component may decrease a magnitude of the glucose cost component if the ROC data indicates that the glucose concentration is increasing and if a difference between the glucose concentration of the user for the current operational cycle and a glucose concentration of the user for a predecessor operational cycle is negative, or if the glucose concentration is decreasing and if a difference between the glucose concentration of the user for the current operational cycle and a glucose concentration of the user for a predecessor operational cycle is positive.

In accordance with an additional inventive facet, an insulin delivery system for delivering insulin to a user includes a non-transitory computer-readable storage medium storing processor-executable instructions and a processor for executing the processor-executable instructions. Executing the instructions causes the processor to receive an ROC reading for an operational cycle of the insulin delivery device from a noninvasive glucose sensor, to determine an offset between the ROC reading and a corresponding subcutaneous glucose sensor reading for the operational cycle relative to one or more prior cycles, and to determine a calibrated subcutaneous glucose sensor reading for the operational cycle using the offset. Executing the instructions also causes the processor to use the calibrated subcutaneous glucose sensor reading to determine an insulin dose for the operational cycle and to cause the insulin dose to be delivered by the insulin delivery device to the user during the operational cycle.

The processor-executable instructions may further cause the processor to calculate an estimate of an ROC of the subcutaneous glucose sensor readings using the ROC reading from the noninvasive sensor for a current operational cycle and an ROC reading from the noninvasive sensor for a predecessor operational cycle. The processor-executable instructions may further cause the processor to determine a value equal to a ratio of a difference between the subcutaneous glucose sensor reading for the current operational cycle and a subcutaneous glucose sensor reading for the predecessor operational cycle and the estimate of an ROC of the subcutaneous glucose sensor readings. The offset may be determined as a difference between the subcutaneous glucose sensor reading for the current operational cycle and a product of the ratio and the ROC reading from the noninvasive sensor for the current operational cycle. The calibrated subcutaneous glucose sensor reading for the operational cycle may be determined by adding the offset to the product of the above-mentioned ratio and the ROC reading from the noninvasive sensor for the current operational cycle. The subcutaneous glucose sensor may be a continuous glucose monitor.

### BRIEF DESCRIPTION OF THE DRAWINGS

FIG. 1A depicts an illustrative medicament delivery system for exemplary embodiments.

FIG. 1B depicts an illustrative block diagram of sensors for exemplary embodiments.

FIG. 2 depicts a flowchart of illustrative steps that may be performed in exemplary embodiments to use glucose rate of change (ROC) values for a user to determine basal insulin delivery rates.

FIG. 3 depicts a flowchart of illustrative steps that may be performed in exemplary embodiments to adjust the basal insulin delivery rate for a time period.

FIG. 4 depicts a flowchart of illustrative steps that may be performed in exemplary embodiments to increase the basal insulin delivery rate for the time period.

FIG. 5 depicts a flowchart of illustrative steps that may be performed in exemplary embodiments to use glucose level ROC values in choosing a basal insulin dose for delivery.

FIG. 6 depicts a flowchart of illustrative steps that may be performed in exemplary embodiments to predict future glucose level ROC values.

FIG. 7 depicts a flowchart of illustrative steps that may be performed in exemplary embodiments to apply the cost function to a candidate insulin dose.

FIG. 8 depicts a flowchart of illustrative steps that may be performed in exemplary embodiments to calibrate the noninvasive glucose sensor with the CGM.

FIG. 9 depicts a flowchart of illustrative steps that may be performed in exemplary embodiments to determine m₀′.

FIG. 10 depicts a flowchart of illustrative steps that may be performed in the exemplary embodiments to determine b₀′.

FIG. 11 depicts a flowchart of illustrative steps that may be performed in exemplary embodiments to realize a substitution of an estimated glucose level value based on a noninvasive glucose sensor reading for a CGM glucose level reading.

FIG. 12 depicts a flowchart of illustrative steps that may be performed to adjust the weight coefficient of the glucose component of the cost function.

FIG. 13 depicts an illustrative table that may be used in exemplary embodiments to adjust a weight coefficient of a glucose cost component of a cost function.

FIG. 14 depicts a flowchart of illustrative steps that may be performed in exemplary embodiments to determine the adjusted weight coefficient for the glucose cost component value of the cost function.

FIG. 15 depicts a flowchart of illustrative steps that may be performed in exemplary embodiments to detect meals using the noninvasive glucose sensor.

FIG. 16 depicts a flowchart of illustrative steps that may be performed in exemplary embodiments to combine glucose level readings from the invasive and noninvasive sensors.

FIG. 17 depicts a flowchart of illustrative steps that may be performed in exemplary embodiments to calculate a trust weight X(k).

FIG. 18 depicts a flowchart of illustrative steps that may be performed in exemplary embodiments to calculate X_{f}(k).

FIG. 19 depicts a flowchart of illustrative steps that may be performed in exemplary embodiments to confirm delivery of a medicament into an interstitial space based on ROC data.

FIG. 20 depicts a flowchart of illustrative steps that may be performed in exemplary embodiments to apply different control laws depending upon the source of glucose data being used by a control method.

FIG. 21 depicts a flowchart of illustrative steps that may be performed in exemplary embodiments to adjust aggressiveness based on trust level.

### DETAILED DESCRIPTION

The exemplary embodiments may use invasive glucose sensors, like CGMs, in conjunction with noninvasive glucose sensors to improve glucose management for a user. For instance, in some exemplary embodiments, ROC of glucose levels from a noninvasive glucose sensor may be used rather than a glucose level of the user from a CGM. The noninvasive sensors do not require a detector to be positioned subcutaneously on the user. A basal insulin delivery rate to the user may be adjusted responsive to the ROC glucose level data from the noninvasive sensor.

In other exemplary embodiments, the glucose level ROC as measured by a noninvasive glucose sensor may be used to predict future glucose level ROCs of the user between operational cycles of an insulin delivery device. These predicted future glucose level ROCs may be used in a cost function of the control system of the insulin delivery device to select basal insulin delivery doses.

Glucose level readings from a CGM may be used to calibrate a noninvasive glucose level sensor. Once the noninvasive glucose sensor is calibrated, the noninvasive glucose level ROC data may be used to estimate a glucose level of the user. In instances in which the glucose level data in not available from the CGM, the estimated glucose level of the user based on the ROC data may be used in place of the glucose level data from the CGM by the control system to determine basal insulin delivery doses.

The glucose level ROC data from the noninvasive glucose sensor may also be used to adjust a weight coefficient of a glucose cost component of a cost function. The adjustment may increase or decrease the magnitude of the weight coefficient. In this fashion, the aggressiveness of the control algorithm relative to glucose excursions may be increased or decreased responsive to the glucose level ROC.

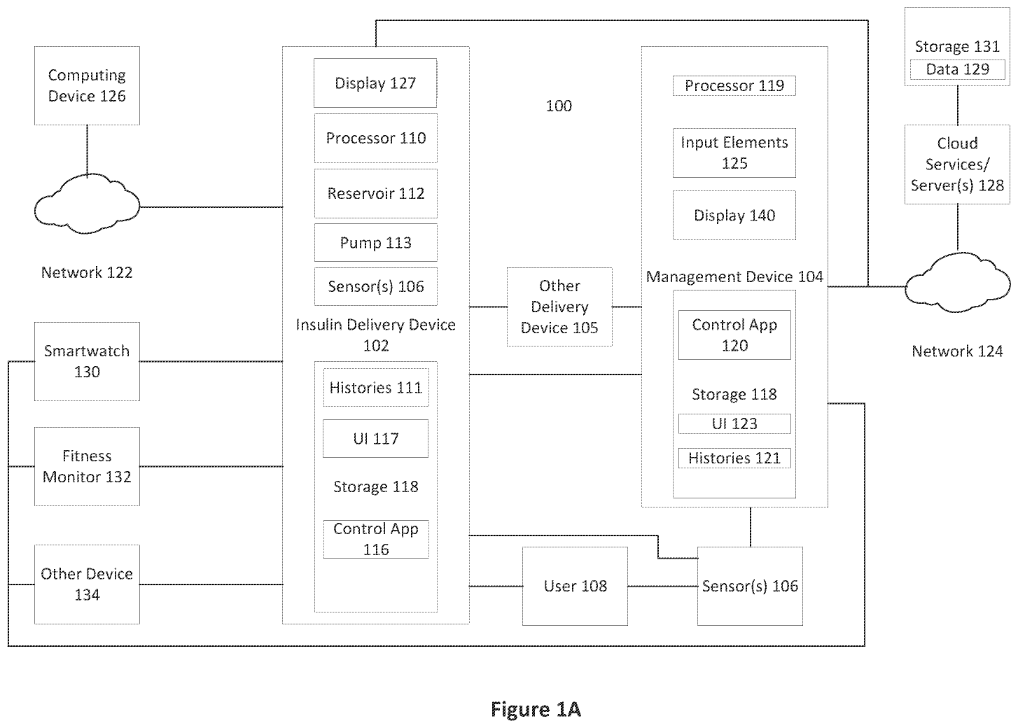

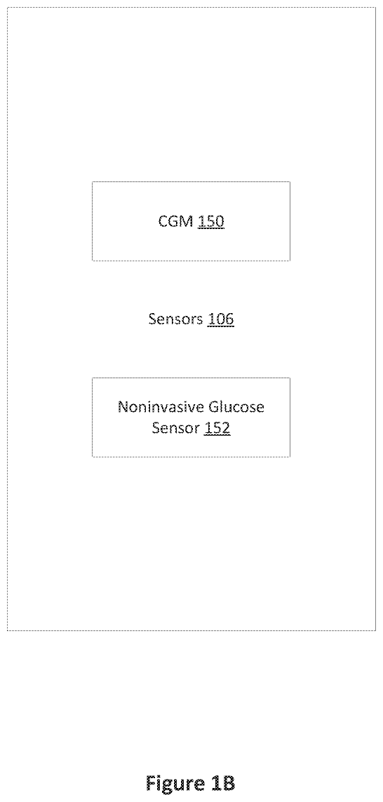

FIG. 1A depicts an illustrative medicament delivery system 100 that is suitable for delivering a medicament to a user 108 in accordance with the exemplary embodiments. The medicament delivery system 100 includes a medicament delivery device 102. The medicament delivery device 102 may be a wearable device that is worn on the body of the user 108 or carried by the user. The medicament delivery device 102 may be directly coupled to a user (e.g., directly attached to a body part and/or skin of the user 108 via an adhesive or the like) with no tubes and an infusion location directly under the medicament delivery device 102, or carried by the user (e.g., on a belt or in a pocket) with the medicament delivery device 102 connected to an infusion site where the medicament is injected using a needle and/or cannula. In a preferred embodiment, a surface of the medicament delivery device 102 may include an adhesive to facilitate attachment to the user 108.

The medicament delivery device 102 may include a processor 110. The processor 110 may be, for example, a microprocessor, a logic circuit, a field programmable gate array (FPGA), an application specific integrated circuit (ASIC) or a microcontroller. The processor 110 may maintain a date and time as well as other functions (e.g., calculations or the like). The processor 110 may be operable to execute a control application 116, which is encoded in computer programming instructions stored in the storage 114, that enables the processor 110 to direct operation of the medicament delivery device 102. The control application implements the control system of the medicament delivery device 102. The control application 116 may be a single program, multiple programs, modules, libraries or the like. The processor 110 also may execute computer programming instructions stored in the storage 114 for a user interface (UI) 117 that may include one or more display screens shown on display 127. The display 127 may display information to the user 108 and, in some instances, may receive input from the user 108, such as when the display 127 is a touchscreen.

The control application 116 may control delivery of a medicament to the user 108 per a control approach like that described herein. The control application 116 may provide the safety and accuracy measures relating to medicament boluses that are described herein. The storage 114 may hold histories 111 for a user, such as a history of basal deliveries, a history of bolus deliveries, and/or other histories, such as a meal event history, exercise event history, glucose level history and/or the like. In addition, the processor 110 may be operable to receive data or information. The storage 114 may include both primary memory and secondary memory. The storage 114 may include random access memory (RAM), read only memory (ROM), optical storage, magnetic storage, removable storage media, solid state storage or the like.

The medicament delivery device 102 may include one or more housings for housing its various components including a pump 113, a power source (not shown), and a reservoir 112 for storing a medicament for delivery to the user 108. Alternatively, the components may be on a tray secured to the body of the patient. A fluid path to the user 108 may be provided, and the medicament delivery device 102 may expel the medicament from the reservoir 112 to deliver the medicament to the user 108 using the pump 113 via the fluid path. The fluid path may, for example, include tubing coupling the medicament delivery device 102 to the user 108 (e.g., tubing coupling a cannula to the reservoir 112) and may include a conduit to a separate infusion site. The medicament delivery device 102 may have operational cycles, such as every 5 minutes, in which basal doses of medicament are calculated and delivered as needed. In some embodiments, the length of a cycle is between about 30 seconds to 20 minutes, more specifically between about 1 min to about 10 min and in particular between about 3 min to 7 min. These steps are repeated for each cycle.

There may be one or more communications links with one or more devices physically separated from the medicament delivery device 102 including, for example, a management device 104 of the user and/or a caregiver of the user, sensor(s) 106, a smartwatch 130, a fitness monitor 132 and/or another variety of device 134. The communication links may include any wired or wireless communication links operating according to any known communications protocol or standard, such as Bluetooth®, Wi-Fi, a near-field communication standard, a cellular standard, or any other wireless protocol.

The medicament delivery device 102 may interface with a network 122 via a wired or wireless communications link. The network 122 may include a local area network (LAN), a wide area network (WAN) or a combination therein. A computing device 126 may be interfaced with the network 122, and the computing device may communicate with the medicament delivery device 102.

The medicament delivery system 100 may include one or more sensor(s) 106 for sensing the levels of one or more analytes. The sensor(s) 106 may be coupled to the user 108 by, for example, adhesive or the like and may provide information or data on one or more medical conditions and/or physical attributes of the user 108. The sensor(s) 106 may be physically separate from the medicament delivery device 102 or may be an integrated component thereof. The sensor(s) 106 may include, for example, glucose monitors, such as continuous glucose monitors (CGM's) and/or noninvasive glucose monitors. The sensor(s) 106 may include ketone sensors, analyte sensors, heart rate monitors, breathing rate monitors, motion sensors, temperature sensors, perspiration sensors, blood pressure sensors, alcohol sensors or the like.

The medicament delivery system 100 may or may not also include a management device 104. In some embodiments, no management device is needed as the medicament delivery device 102 may manage itself. The management device 104 may be a special purpose device, such as a dedicated personal diabetes manager (PDM) device. The management device 104 may be a programmed general-purpose device, such as any portable electronic device including, for example, a dedicated controller, such as a processor, a micro-controller, or the like. The management device 104 may be used to program or adjust operation of the medicament delivery device 102 and/or the sensor(s) 106. The management device 104 may be any portable electronic device including, for example, a dedicated device, a smartphone, a smartwatch or a tablet. In the depicted example, the management device 104 may include a processor 119 and a storage 118. The processor 119 may execute processes to manage a user's glucose levels and to control the delivery of the medicament to the user 108. The medicament delivery device 102 may provide data from the sensors 106 and other data to the management device 104. The data may be stored in the storage 118. The processor 119 may also be operable to execute programming code stored in the storage 118. For example, the storage 118 may be operable to store one or more control applications 120 for execution by the processor 119. The control application 120 may be responsible for controlling the medicament delivery device 102, such as by controlling the automated insulin delivery (AID) of insulin to the user 108. In some exemplary embodiments, the control application 120 provides the adaptability described herein. The storage 118 may store the control application 120, histories 121 like those described above for the medicament delivery device 102, and other data and/or programs.

A display 140, such as a touchscreen, may be provided for displaying information. The display 140 may display user interface (UI) 123. The display 140 also may be used to receive input, such as when it is a touchscreen. The management device 104 may further include input elements 125, such as a keyboard, button, knobs, or the like, for receiving input form the user 108.

The management device 104 may interface with a network 124, such as a LAN or WAN or combination of such networks, via wired or wireless communication links. The management device 104 may communicate over network 124 with one or more servers or cloud services 128. Data, such as sensor values, may be sent, in some embodiments, for storage and processing from the medicament delivery device 102 directly to the cloud services/server(s) 128 or instead from the management device 104 to the cloud services/server(s) 128.

Other devices, like smartwatch 130, fitness monitor 132 and device 134 may be part of the medicament delivery system 100. These devices 130, 132 and 134 may communicate with the medicament delivery device 102 and/or management device 104 to receive information and/or issue commands to the medicament delivery device 102. These devices 130, 132 and 134 may execute computer programming instructions to perform some of the control functions otherwise performed by processor 110 or processor 119, such as via control applications 116 and 120. These devices 130, 132 and 134 may include displays for displaying information. The displays may show a user interface for providing input by the user, such as to request a change or pause in dosage or to request, initiate, or confirm delivery of a bolus of a medicament, or for displaying output, such as a change in dosage (e.g., of a basal insulin delivery amount) as determined by processor 110 or management device 104. These devices 130, 132 and 134 may also have wireless communication connections with the sensor 106 to directly receive analyte measurement data. Another delivery device 105, such as a medicament delivery pen, may be provided for also delivering medicament to the user 108. The other device 105 is referred to herein as a secondary medicament delivery device or auxiliary device, whereas the medicament delivery device 102 is referred to as the primary medicament delivery device.

A wide variety of medicaments may be delivered by the medicament delivery device 102 and delivery device 105. The medicament may be insulin for treating diabetes and for purposes of the exemplary embodiments described herein, it is presumed that the medicament delivery device delivers insulin and may deliver other medicaments as well in some exemplary embodiments. The medicament also may be glucagon for raising a user's glucose level. The medicament may be a glucagon-like peptide (GLP)-1 receptor agonists for lowering glucose or slowing gastric emptying, thereby delaying spikes in glucose after a meal. Alternatively, the medicament delivered by the medicament delivery device 102 may be one of a pain relief agent, a chemotherapy agent, an antibiotic, a blood thinning agent, a hormone, a blood pressure lowering agent, an antidepressant, an antipsychotic, a statin, an anticoagulant, an anticonvulsant, an antihistamine, an anti-inflammatory, a steroid, an immunosuppressive agent, an antianxiety agent, an antiviral agents, a nutritional supplement or a vitamin. The medicament may be a coformulation of two or more of those medicaments listed above.

The functionality described herein for the exemplary embodiments may be under the control of or performed by the control application 116 of the medicament delivery device 102 or the control application 120 of the management device 104. In some embodiments, the functionality wholly or partially may be under the control of or performed by the cloud services/servers 128, the computing device 126 or by the other enumerated devices, including smartwatch 130, fitness monitor 132 or another wearable device 134.

In the closed loop mode, the control application 116, 120 determines the medicant delivery amount for the user 108 on an ongoing basis based on a feedback loop. For an insulin delivery device, the aim of the closed loop mode is to have the user's glucose level at a target glucose level. The target glucose level of a user may be a certain value, e.g. 110 mg/dL, or may be a range, e.g. about 90 mg/dL to about 130 mg/dL.

As was mentioned above various types of sensors 106 may be used in the medicament delivery system 100. FIG. 1B depicts a subset of the sensors 106 that may be used in the medicament delivery system 100 when the medicament is insulin and the medicament delivery device 102 is an insulin delivery device. The sensors 106 shown in FIG. 1B include a CGM 150 that is worn on-body by the user 108 and that includes a sensor element that is positioned subcutaneously. The CGM 150 may be characterized as an “invasive sensor.” The sensors 106 shown in FIG. 1B also include a noninvasive glucose sensor 152. The term “invasive (glucose) sensor” as used herein may relate to a sensor that requires a break in the skin to operate, i.e. to measure an analyte value, in particular the blood glucose value. The term “non-invasive (glucose) sensor” as used herein may relate to a sensor that does not require a break in the skin to operate, i.e. to measure an analyte value, in particular the blood glucose value. An example of a noninvasive glucose sensor is a sensing wristband that detects ROC of glucose level using photonics technology, a commercial example of which is the Rockley Bioptx sensing wristband. The wristband emits laser lines over a wide range of wavelengths and processes the reflection/absorption to determine ROC of a glucose level of a user. Other noninvasive glucose sensors may be used in exemplary embodiments. The reference herein to a sensing wristband is illustrative and not intended to be limiting.

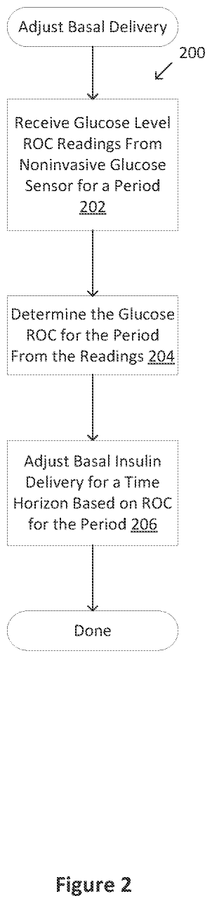

As will be detailed below, the CGM 150 and the noninvasive glucose sensor 152 may be used in conjunction in some exemplary embodiments to improve glucose management for the user 108. Also, the noninvasive glucose sensor 152 may be used instead of the CGM 150 in some instances. For example, in some exemplary embodiments, glucose ROC data from a noninvasive sensor 152 may be used in determining basal insulin delivery dosages for a time period rather than a CGM 150, as is conventionally used. FIG. 2 depicts a flowchart 200 of illustrative steps that may be performed in exemplary embodiments to use glucose level ROC values for a user 108 rather than glucose level values to determine basal insulin delivery rates in an insulin delivery system. It is presumed, for the example, that the control system of the medicament delivery device 102 is not receiving glucose level values from a CGM at periodic intervals, such as every five minutes but rather is receiving glucose level ROC data from a noninvasive glucose sensor 152 at periodic intervals, such as every five minutes. At 202, the control application 116 or 120 receives glucose level ROC readings from the noninvasive glucose sensor 152 for a time period, such as a 30 minute period. The glucose level ROC data may indicate a magnitude of change and whether the change is positive or negative. At 204, the glucose level ROC for the time period is determined. The ROC for the 30 minute period is the sum of the glucose level ROC readings from the noninvasive glucose sensor 152 for the period. At 206, the basal insulin delivery to the user 108 by the medicament delivery device 102 for a time horizon (e.g., 90 minutes) is adjusted based on the glucose ROC for the period as is detailed below. Hence, the glucose ROC readings may be used to set glucose dosages for the user for the time horizon without the need for glucose level readings from a CGM. This may be beneficial to the user 108 as the noninvasive glucose sensors may be cheaper, more convenient and less cumbersome than conventional invasive glucose sensors, like CGMs.

The steps of the flowchart 200 of FIG. 2 may be repeated at regular intervals, such as every thirty minutes, or at changing intervals, such as at longer intervals at night when a user likely is sleeping. Alternatively, the steps may be triggered by events or by user request.

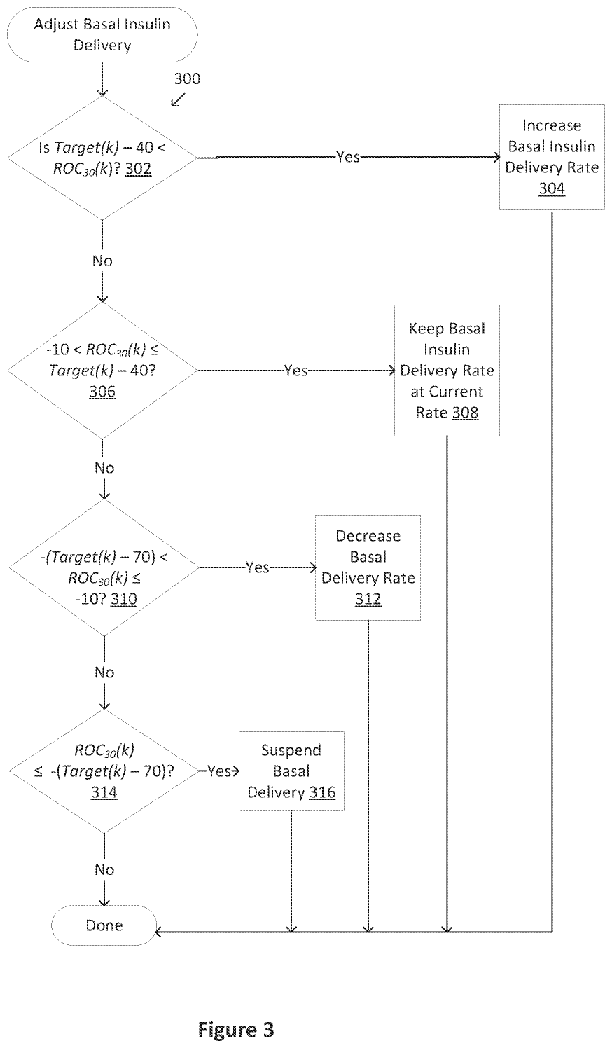

FIG. 3 depicts a flowchart 300 of illustrative steps that may be performed in exemplary embodiments to adjust the basal insulin delivery rate for the time horizon (see 206 in FIG. 2). At 302, a check is made whether the target glucose level for the time horizon, Target(k), minus 40 is less than the ROC for the past 30 minutes, ROC₃₀(k). The idea of this check is that the target glucose level (e.g., 110 mg/dL) minus the difference relative to the hyperglycemic threshold (e.g., 150 mg/dL) constitutes how much the glucose level of the user 108 can increase before the user 108 becomes hyperglycemic. If the deviation over the time window ROC₃₀(k) exceeds 40 mg/dL, there is a likelihood that the user 108 will become hyperglycemic if the trend continues. Hence, in that instance, at 304, the basal insulin delivery rate is increased to avoid the hyperglycemia.

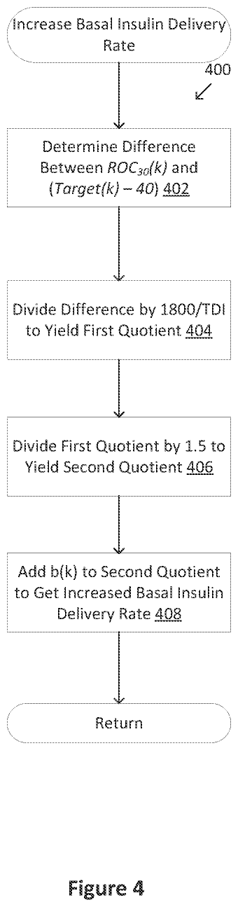

A suitable equation for calculating the increase in basal insulin delivery rate based on ROC is:

$${b}_{increase} ( k ) = \frac{( R O {C}_{30} ( k ) - ( Target ( k ) - 40 ) )}{\frac{1800}{T D I}} \cdot \frac{1}{1.5} + b ( k ) \qquad ( Eq . 1 )$$

where b_{increase}(k) is the increase in the basal rate (i.e., dose every cycle) and TDI is the total daily insulin for the user 108. The basal insulin delivery rate is expressed as an hourly rate in Equation 1. FIG. 4 depicts a flowchart 400 of illustrative steps that may be performed in exemplary embodiments to increase the basal insulin delivery rate for the time horizon based using Equation 1. At 402, the difference (ROC₃₀(k)−(Target(k)−40)) is calculated, where 40 represents the difference in glucose concentrations between a typical target glucose value of 110 mg/dL and a typical hypoglycemic threshold of 70 mg/dL. The difference between the glucose target value and the hypoglycemic threshold may also be expressed as “Δ_{hypoglycemia}” and may vary from the value of 40 mg/dL, for example if the user has a different target than 110. The basal insulin delivery rate may be increased to bring the glucose level back down into an acceptable range level since the ROC₃₀(k) is high enough to ensure that there is minimal risk of hypoglycemia for the time horizon. At 404, the difference is divided by the correction factor of 1800/TDI to convert the difference into an amount of insulin needed to compensate for the difference. 1800 is a tunable factor and may also be expressed as “A”. At 406, the resulting quotient is divided by 1.5 to make it an hourly rate value since the insulin is delivered over a 90 minute time (i.e., 1.5 hours). The length of insulin delivery may also be expressed as “LID”. At 408, the resulting hourly rate value is added to the currently specified basal insulin delivery rate for the time horizon to determine the increased basal insulin delivery rate. Accordingly, the above equation Eq.1 may be formulated as:

$${b}_{increase} ( k ) = \frac{( R O {C}_{30} ( k ) - ( {Δ}_{hypoglyemia} ( k ) ) )}{\frac{A}{T D I}} \cdot \frac{60}{L I D} + b ( k ) \qquad ( Eq . 1 a )$$

It should be understood that the specific value of “minus 40” (−40), the target glucose level of 110 mg/dL, the hyperglycemic threshold of 70 mg/dL, the hypoglycemic threshold of 70 mg/dL, the correction factor of 1800/TDI and the value of 1.5 (for 1.5 hours) mentioned in context with equation 1 (and also below) are only exemplary values, whereas the present invention is not limited to these exemplary values. For example, the value of minus 40 may alternatively be a value ranging from minus 20 to minus 60, in particular from minus 30 to minus 50. In addition, the target glucose level may range from 90 to 130 mg/dL, in particular from 100 to 120 mg/dL. The hypoglycemic threshold may range from 60 to 80 mg/dL. The hyperglycemic threshold may range from 140 to 160 mg/dL. The correction factor may range from 300/TDI to 5000/TDI, in particular 900/TDI to 2700/TDI. And the specific value of 1.5 may actually range from 0.5 to 3, in particular from 1 to 2.

With reference to FIG. 3, if (Target(k)−40) is not less than ROC₃₀(k) as checked at 302, at 306, a check is made whether ROC₃₀(k) is greater than −10 and less than or equal to (Target(k)−40). The “−10” may constitute a first negative threshold value and may be a tunable factor. In some embodiments, the first negative threshold value is between about −3 to about −20, more specifically between about −5 to about −15 and in particular between about −7 to about −13. Based on the target glucose level of 110 mg/dL and a hyperglycemic threshold of 150 mg/dL, the check at 406 determines whether the ROC₃₀(k) is in a range between −10 and 70. In that case, the glucose level of the user 108 is in an acceptable range and at 308, the basal insulin delivery rate is kept as the current rate.

If at 306, it is determined that ROC₃₀(k) is not greater than −10 or not less than or equal to (Target(k)−40), at 310, a check is made whether the basal insulin delivery rate should be decreased. Specifically, a check is made whether ROC₃₀(k) is greater than −(Target(k)−70) but less than or equal to −10. In this illustrative formulation, the hypoglycemic threshold is 70 mg/dL, and the target is 110 mg/dL; so −(Target(k)−70) is −40 mg/dL. Hence, the check determines whether the ROC in the next 30 minutes (assuming the same trend) may cause the user to be hypoglycemic and whether the ROC₃₀(k) is in the range of −10 mg/dL to −40 mg/dL. The 30 minute time frame is just exemplary, and may also be varied, e.g. between about 20 mins to about 60 mins. If so, at 312, the basal insulin delivery rate is decreased, such as by cutting the basal insulin delivery rate in half Again, −40 mg/dL may constitute a second negative threshold value and may also be tunable factor. In some embodiments, the second negative threshold value is between about −25 to about −60, more specifically between about −30 to about −50 and in particular between about −35 to about −45. In some embodiments, the basal insulin delivery rate is decreased between about 10% to about 80%, more specifically between about 30% to about 70% and in particular between about 40% to about 60% in response to the check determining that the ROC in a coming time frame may cause the user to be hypoglycemic and that the ROC₃₀(k) is in the range between the first negative threshold and the second negative threshold. If not, at 314, a check is made whether ROC₃₀(k) is less than or equal to −(Target(k)−70) (i.e., −40 mg/dL). If so, at 316, the basal insulin delivery is stopped because there is a substantial risk of hypoglycemia if the trend continues.

It should be appreciated that Target(k) may be set at values other than 110 mg/dL. In addition, the increase in basal insulin delivery rate may be calculated in other fashions. Similarly, the decrease in basal insulin delivery rate may be set at an amount other than a fifty percent decrease. The decrease could be forty percent or sixty percent, for instance. Alternately, the decrease in basal delivery also may be calculated in a similar manner as Equation 1 with proportional changes based on the ROC₃₀ values. The hypoglycemic threshold and the hyperglycemic thresholds may also be set at different values than those detailed above.

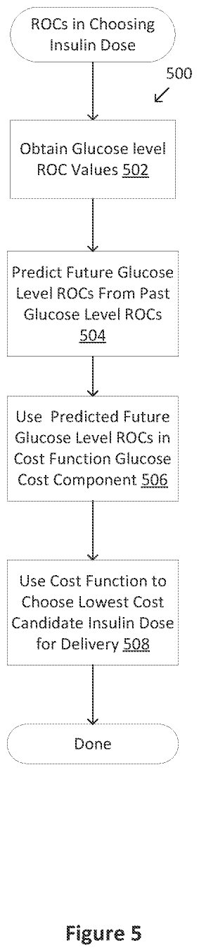

In other exemplary embodiments, the ROCs of glucose level of the user 108 between operational cycles may be incorporated into predicting future deviations and in a cost function for selecting basal insulin doses for delivery to the user 108. A glucose deviation for a cycle may be determined as the difference in glucose level at the current cycle and the glucose level at the previous cycle. Such a glucose deviation represents a glucose level ROC over for example a five minute time, i.e. if the length of a cycle is 5 minutes. FIG. 5 depicts a flowchart 500 of illustrative steps that may be performed in exemplary embodiments to use glucose deviations in choosing a basal insulin dose for delivery. At 502, the glucose level ROC values are obtained from a noninvasive glucose sensor 152. These glucose level ROC values may be obtained for each operational cycle of a time period from the noninvasive glucose sensor 152. Each ROC value has a sign (either positive or negative) and a magnitude. For example, if the glucose level of the user 108 in the previous cycle was 110 and the glucose level of the user in the current cycle is 115, the ROC would be +5.

At 504, future glucose level ROCs for the user 108 are predicted from past glucose level ROCs. The notion is that the glucose level ROC trend can be used to predict future level ROCs. At 506, the predicted future glucose level ROCs may be incorporated into a glucose cost component of the cost function for candidate insulin doses. At 508, the cost function is used to choose the lowest cost basal insulin dose, e.g. the insulin dose that results in a minimum output of the cost function, for delivery by the medicament delivery device 102 to the user 108.

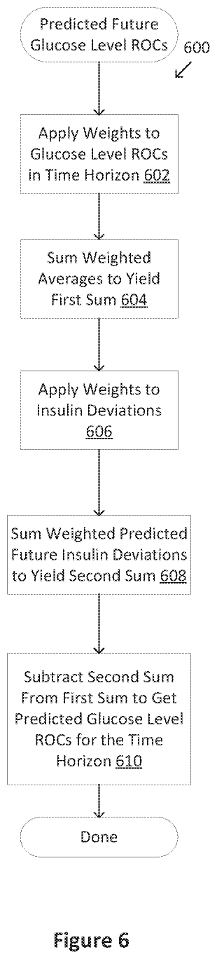

FIG. 6 depicts a flowchart 600 of illustrative steps that may be performed in exemplary embodiments to predict future glucose level ROCs (see 504 in FIG. 5). A suitable equation for determining the glucose deviation for time window is:

G′(k)=b₁G′(k−1)+b₂G′(k−2)+ . . . bₙG′(k−n)−K₁I(k−1)−K₂I(k−2) . . . −KₘI(k−m) (Eq. 2)

where k is a cycle index, G′(k) is the glucose deviation relative to a preceding cycle, I(k) is the insulin dose deviation of the basal insulin does for cycle k and a baseline insulin dose, and b and K are weight coefficients.

Initially at 602, the weight coefficients b are applied to the glucose level ROC values in the predicted time horizon. At 604, these weighted glucose level ROC values are summed (i.e., b₁G′(k−1)+b₂G′(k−2)+ . . . bₙG′(k−n)) to yield a first sum. At 606, weight coefficients are applied to the insulin deviation values, and at 608 the weighted insulin deviation values are summed to produce a second sum as will be detailed below. At 610, the second sum is subtracted from the first sum to produce the predicted glucose level ROC of G′(k).

As mentioned above, the predicted glucose level ROCs may be used in the cost function rather than glucose level values (see 506 in FIG. 5). A suitable cost function is:

J(k)=Q·Σᵢ₌₁^{M}|Gₖ′(i)|²+R·Σᵢ₌₁^{N}Iₖ(i)|² (Eq. 3)

where Q is a glucose cost weight coefficient, R is an insulin cost weight coefficient, M is the number of cycles in a time horizon, Nis a number of cycles in a time horizon, i is a cycle index, and Iₖ(i) is an insulin deviation between a predicted insulin dose for cycle i and a baseline insulin dose.

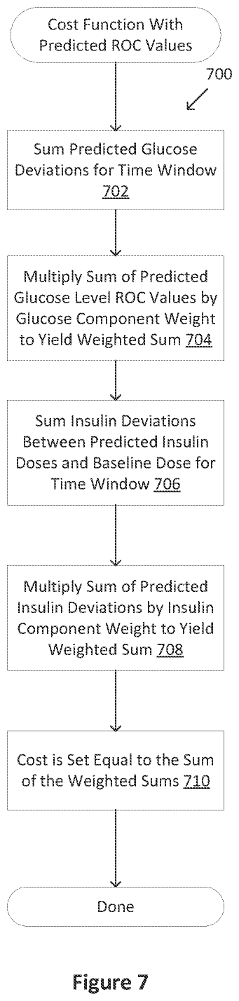

FIG. 7 depicts a flowchart 700 of illustrative steps that may be performed in exemplary embodiments to apply the cost function to a candidate insulin dose. At 702, the predicted glucose level ROC values for a time window are summed (i.e., Σᵢ₌₁^{M}|Gₖ′(i)|²). At 704, the sum of predicted glucose level ROC values for the time window is multiplied by the glucose weight coefficient Q. At 706, the insulin deviations between the predicted insulin doses and the baseline dose for cycles in the time window are summed (i.e., Σᵢ₌₁^{N}|Iₖ(i)|²). At 708, this sum is multiplied by the weight coefficient R of the insulin cost. At 710, the weighted sums are added to yield the cost.

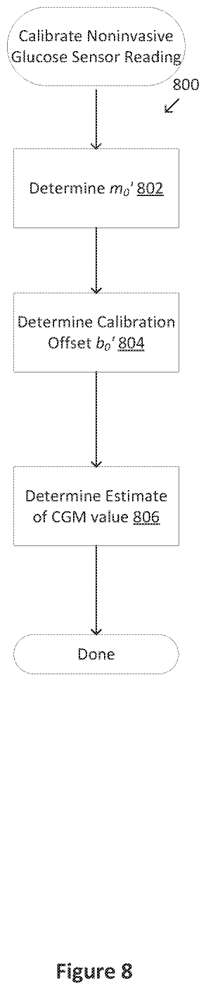

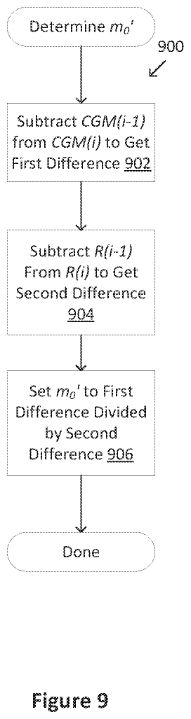

The noninvasive glucose sensor 152 may be used in conjunction with a CGM 150. Specifically, the CGM 150 may be used to calibrate the noninvasive glucose sensor 152. The calibrated noninvasive sensor 152 then may be used in place of the CGM when readings from the CGM are unavailable. FIG. 8 depicts a flowchart 800 of illustrative steps that may be performed in exemplary embodiments to calibrate the noninvasive glucose sensor 152 with the CGM. At 802, m₀′ is determined. The value m₀′ is the slope when the estimated CGM value is estimated from the noninvasive glucose sensor value in slope intercept from in Equation 11 below. FIG. 9 depicts a flowchart 900 of illustrative steps that may be performed in exemplary embodiments to determine m₀′. As an initial matter, the noninvasive glucose sensor 152 may convert a signal value R(i) into a glucose level value G₀(i) that is not visible to a user. This relationship may be expressed as G₀(i)=m₀R(i)+b₀(Eq. 4), where G₀(i) is the estimated glucose level reading for cycle i, R(i) is the ROC reading from noninvasive glucose sensor 152, m₀ is a correlation value, and b₀ is an offset value. In some embodiments, the offset value is not known to the noninvasive sensor 152.

The ROC for the CGM glucose level readings may be expressed as: ROC₀(i)=G₀(i)−G₀(i−1) (Eq. 5). Substituting equivalents based on Equation 4 yields ROC₀(i)=(m₀R(i)+b₀)−(m₀R(i−1)+b₀) (Eq. 6), which can then be simplified as ROC₀(i)=m₀(R(i)−R(i−1)) (Eq. 7). Then, this equation may be used to solve for m₀ as

$${m}_{0} = \frac{R O {C}_{0} ( i )}{R ( i ) - R ( i - 1 )} = \frac{{G}_{0} ( i ) - {G}_{0} ( i - 1 )}{R ( i ) - R ( i - 1 )} . \qquad ( Eq . 8 )$$

The readings from the CGM 150 may be substituted for the estimated glucose level readings G₀ as follows:

$${m}_{0}^{′} = \frac{C G M ( i ) - C G M ( i - 1 )}{R ( i ) - R ( i - 1 )} . \qquad ( Eq . 9 )$$

Thus, at 902, CGM(i)−CGM(i−1) is calculated to get a first difference. At 904, the deviation in successive noninvasive glucose sensor 154 values is determined as R(i)−R(i−1) to determine a second difference. At 906, the first difference is divide by the second difference to yield m₀′.

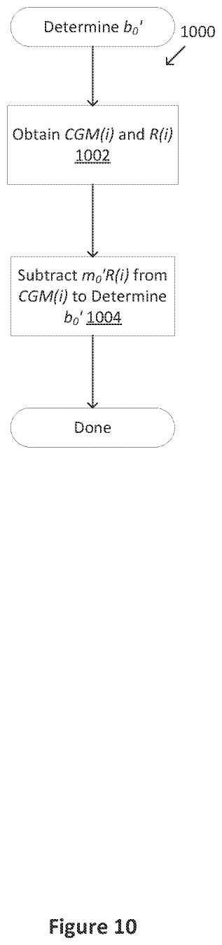

The calibration continues at 804, in determining the calibration offset b₀′. FIG. 10 depicts a flowchart 1000 of illustrative steps that may be performed in the exemplary embodiments to determine b₀′. At 1002, the CGM(i) value is obtained from CGM 150 and R(i) value is obtained from noninvasive glucose sensor 152. At 1004, m₀′R(i) is subtracted from CGM(i) to determine the calibration offset b₀′. Hence the calibration offset is calculated as

b₀′=CGM(i)−m₀′R(i) (Eq.10)

At 806, the estimate of the glucose level reading, designated as CGM′(i), may be calculated from the noninvasive sensor reading as follows:

CGM′(i)=m₀′R(i)+b₀′ (Eq. 11).

This calculation may be performed, for example, each operational cycle or at other intervals.

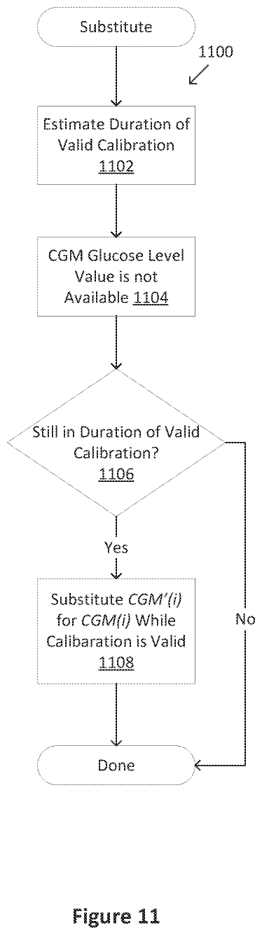

As was mentioned above, the estimate of the glucose level reading from the CGM 150 may be substituted for the CGM glucose level reading when the CGM glucose level reading is not available. FIG. 11 depicts a flowchart 1100 of illustrative steps that may be performed in exemplary embodiments to realize such a substitution. At 1102, an estimate of a duration of valid calibration is determined. For example, historical data may be reviewed to estimate the number of cycles before the calculated glucose level values derived from the noninvasive glucose sensor 152 readings significantly diverge (e.g., by more 10%) from the glucose level readings of the CGM 150. In some embodiments, the threshold for determining that the calculated glucose level values derived from the noninvasive glucose sensor 152 readings significantly diverge from the glucose level readings of the CGM 150 is between about 3% to about 30%, more specifically between about 5% to about 20% and in particular between about 7% to about 15%. Alternatively, the duration may be determined based on the stability of the calibrated slope and intercept values, m₀′ and b₀. For instance, the calibration may be valid until the slope an intercept values diverge more than a threshold amount, like 20%. In some embodiments, the threshold for determining that the calibration is not valid anymore is between about 5% to about 40%, more specifically between about 10% to about 30% and in particular between about 15% to about 25%. Further, alternatively, the function between the raw sensor readings from the noninvasive sensor and the expected CGM readings may be estimated in different formulations, such as a quadratic form, or others, with corresponding identification of the best fit parameters based on available CGM reading and raw sensor readings.

At 1104, a glucose level reading is not available from the CGM 150. For example, a new CGM may be in the process of being installed, there may be a communication link issue, or other interruption. At 1106, a check is made whether the current cycle is within the duration of valid calibration. If so, at 1108, CGM′(i) is substituted for CGM(i). If not, there is no substitution.

The noninvasive sensor 152 also may be used in conjunction with the CGM 150 to improve the effectiveness of glucose management by the medicament delivery device 102. In particular, the ROC values may be used to help adjust the weight coefficient of the glucose cost component that is used in determining a best insulin dose for the user 108. The weight coefficient may be adjusted to increase the weight to be more aggressive in reducing glucose excursions or to decrease the weight to be less aggressive in reducing glucose excursions.

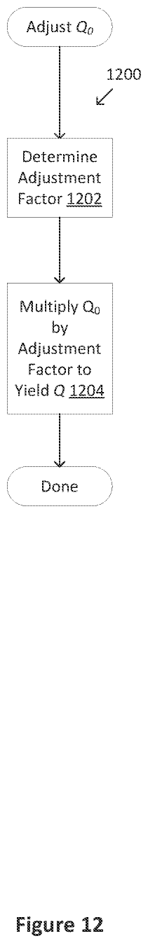

FIG. 12 depicts a flowchart 1200 of illustrative steps that may be performed to adjust the weight coefficient of the glucose component of the cost function. The cost function may be expressed as

J(t)=Q·Σᵢ₌₁^{M}(CGM(i)−target(i))²+R·Σᵢ₌₁^{N}(Iₚ(i)−I_{b}(i)² (Eq.12)

where J(t) is the insulin cost for candidate insulin dose t, Iₚ(i) is the predicted insulin dose for cycle i, and I_{b}(i) is baseline insulin dose for cycle i. At 1202, an adjustment factor is determined based in part on the ROC reading from the noninvasive glucose sensor for cycle I. At 1204, the current weight coefficient Q₀ for the glucose cost component Σᵢ₌₁^{M}(CGM(i)−target(i)) is adjusted by multiplying it by the adjustment factor to produce an adjusted value Q.

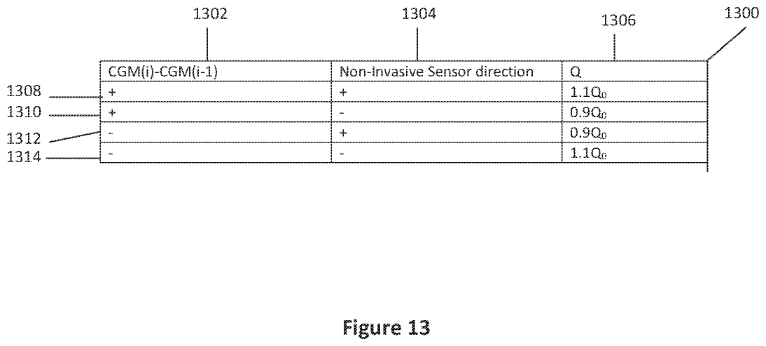

A first option for adjusting the weight coefficient Q is to apply the logic of a table like that of table 1300 shown in FIG. 13. Column 1302 hold indications of whether the deviation between the CGM 150 glucose level reading for i and the CGM 150 glucose reading for cycle i−1 is positive (+) or negative (−). Column 1304 holds an indication of whether the ROC value from the noninvasive glucose sensor 152 is positive (+) or negative (−). Column 1306 holds values that specify the adjusted value of Q relative to the current value of Q, designated as Q₀, given the indicated conditions of columns 1302 and 1304. Thus, when the deviation of CGM values is positive and the noninvasive glucose sensor ROC is positive, as in row 1308, the adjustment factor is 1.1 so that the weight of Q is increased to reduce positive glucose excursions because both conditions of columns 1302 and 1304 indicate an increasing glucose trend. In contrast, if one of the columns 1302 or 1304 is positive and one is negative as in rows 1310 and 1312, the adjustment factor is 0.9 as indicated in column 0.9 to decrease the value of Q. When the deviation of CGM values is negative and the noninvasive glucose sensor ROC is negative as in row 1308, the adjustment factor is 1.1 so that the weight of Q is increased to reduce negative glucose excursions because both conditions of columns 1302 and 1304 indicate a decreasing glucose trend. The “1.1” and “0.9” of FIG. 13 are exemplary factors and may vary.

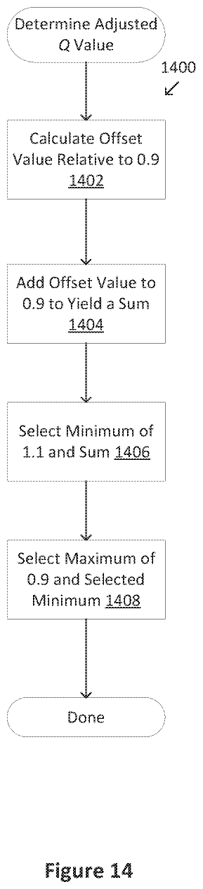

An alternative is to apply a formula to determining the adjustment factor. A suitable formula is

$$Q = {Q}_{0} \cdot max ( min ( 1.1 , 0.9 + \frac{0.1}{\vert ( C G M ( i ) - C G M ( i - 1 ) ) - R O {C}_{NI} \vert} ) , 0.9 ) \qquad ( Eq . 13 )$$

where max is a function that returns a maximum, min is a function that return a minimum, and ROC_{NI} is the rate of change value for cycle i from the noninvasive glucose sensor 152. FIG. 14 depicts a flowchart 1400 of illustrative steps that may be performed in exemplary embodiments to the adjusted Q value. At 1402, the offset value relative 0.9 is calculated. The offset value is calculated as

$$\frac{0.1}{\vert ( C G M ( i ) - C G M ( i - 1 ) ) - R O {C}_{NI} \vert} .$$

The offset value is inversely proportional to the magnitude of the difference between (CGM(i)−CGM(i−1)) and ROC_{NI}. At 1404, the offset value is added to 0.9. At 1406, the minimum of 1.1 and the sum is selected. At 1408, the maximum of 0.9 and the selected minimum is chosen as the adjustment factor that is used at 1204 to get the adjusted value of Q. It is worth noting that the thresholds for modifications of +10% and −10% to the Q₀ value as listed in equation 13 is a tuning parameter that may be modified based on the variations in maximum confidence in the ROC values or other factors. In some embodiments, the thresholds for modifications may be between about +30% and about −30%, more specifically between about +20% and about −20% and in particular between about +10% and about −10%. The absolute value of the threshold for modifications may be expressed as “B”. Accordingly, equation 13 may be expressed as:

$$Q = {Q}_{0} \cdot max ( min ( 1 + B ) , ( 1 - B ) + \frac{B}{\vert ( C G M ( i ) - C G M ( i - 1 ) ) - R O {C}_{NI} \vert} ) , ( 1 - B ) ) \qquad ( Eq . 13 a )$$

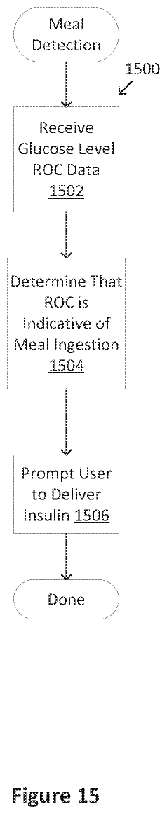

The noninvasive glucose sensor 152 may detect glucose trends more quickly than the CGM 150. Latency in CGM measurement responsive to venous glucose changes may be around 5 minutes. In the 5 minute period, the glucose level of the user 108 may rise 2 mg/dL. The noninvasive glucose sensor 152 may detect glucose level rises much more quickly. For instance, in some exemplary embodiments, the noninvasive glucose sensor 152 may be used to detect meals. Further the noninvasive glucose sensor 152 may be used to identify potentially imminent hypoglycemic events or potentially imminent hyperglycemic events more quickly than with a CGM. A hypoglycemic event or hyperglycemic event as referred to herein may relate to the blood glucose level of the user exceeding a hypoglycemic or hyperglycemic threshold, respectively. FIG. 15 depicts a flowchart of illustrative steps that may be performed in exemplary embodiments to detect meals using the noninvasive glucose sensor 152. At 1502, the control application 116 or 120 receives glucose level ROC data from the noninvasive glucose sensor. The noninvasive glucose sensor may be activated by the user responsive to a meal event or anticipated meal event or may triggered by a calendar of typical meal times. At 1504, the glucose level ROC data is processed to see if it is indicative of a rapid rise associated with meal ingestion. The rise indicated by the glucose level ROC data may be compared to a threshold, for instance, to determine if a meal likely was ingested. In response to the detection of the rapid rise, at 1506, the user 108 may be prompted to take insulin, such as ultra-rapid insulin via nasal spray or other rapid delivery mechanism to respond to the meal ingestion and corresponding rapid rise in glucose level. The prompt may be displayed on display 127 or 140 to inform the user of the need to take insulin.

In some exemplary embodiments, the invasive glucose sensor glucose level readings and noninvasive glucose sensor glucose level readings may be combined to produce combined glucose level values that may be used by the control application is determining basal insulin doses. Noninvasive glucose sensors may produce glucose level readings that conform to an acceptable level of accuracy under normal conditions. However, the readings may become significantly worse under circumstances, such as a rapid rate of change in glucose level of a user. In such circumstances, the exemplary embodiments may rely upon the glucose level readings from an invasive glucose sensor, like a CGM, to be combined with the glucose level readings from the noninvasive glucose sensor to compensate for the deterioration in accuracy of the noninvasive glucose sensor.

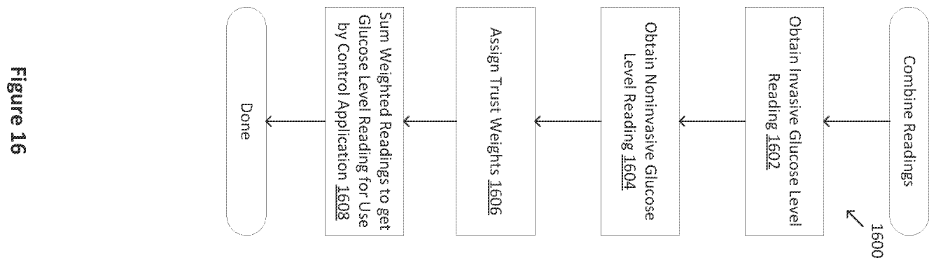

FIG. 16 depicts a flowchart 1600 of illustrative steps that may be performed in exemplary embodiments to combine the glucose level readings to produce a more accurate reading for use by the control application 116 or 120 in controlling delivery of insulin to the user by the insulin delivery device 102. At 1602, a glucose level reading is obtained from the invasive glucose sensor (e.g., CGM 150) for a cycle of the insulin delivery device. At 1604, a corresponding glucose level reading from the noninvasive glucose sensor 152 for the cycle is obtained. These glucose level readings may be combined in some exemplary embodiments by applying the following equation:

Gᵢₙₚᵤₜ(k)=(1−X(k))G_{CGM}(k)+X(k)G_{NI}(k) (Eq. 14)

where Gᵢₙₚᵤₜ(k) is the combined glucose level value, k is the cycle number, G_{CGM}(k) is the glucose level reading from the invasive glucose sensor for cycle k, G_{NI}(k) is the glucose level reading from the noninvasive glucose sensor for cycle k, and X(k) is the trust weight. Thus, at 1606, trust weights X(k) and 1−X(k) are assigned. X(k) is a weight that reflects the level of trust to be provided to glucose level readings from the noninvasive glucose sensor. X(k) may have a value in the range between 0 and 1. 1−X(k) is the weight given to the glucose level reading from the invasive glucose sensor when combining the glucose level readings. In many instances, X(k) has a value between 0 and 0.2. The value X(k) may be based for example on empirical data or calculated as shown below. At 1608, the glucose level reading for use by the control application 116 or 120 is obtained as the sum of the weighted glucose level readings.

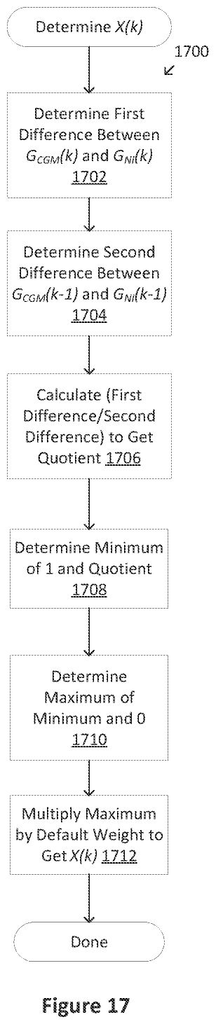

FIG. 17 depicts a flowchart 1700 of illustrative steps that may be performed in exemplary embodiments to determine X(k), where the maximum value X(k) may assume is 0.2 and the minimum value X(k) may assume is 0. A suitable equation for calculating X(k) is:

X(k)=0.2 max (0,min (1,(G_{CGM}(k)−G_{NI}(k))/(G_{CGM}(k−1)−G_{NI}(k−1)))) (Eq. 15).

Hence, at 1702, a first difference G_{CGM}(k)−G_{NI}(k) is determined. The first difference captures the difference in the glucose level reading between the invasive glucose sensor and the noninvasive glucose sensor for the current cycle k. At 1704, a second difference G_{CGM}(k−1)−G_{NI}(k−1) is determined. The second difference captures the difference in the glucose level reading between the invasive glucose sensor and the noninvasive glucose sensor for the predecessor cycle k−1. At 1706, the quotient of the first difference and the second difference is calculated. The quotient captures how the difference in glucose level readings between the sensors is trending. At 1708, a minimum of 1 and the quotient is determined. Thus, if the second difference is larger than the first difference, the quotient is chosen, and 1 is chosen otherwise. At 1710, the maximum of 0 and 1710 is chosen to eliminate possible negative weights. At 1712, the quotient is multiplied with the default weight (i.e., 0.2 in this case) to determine the value of X(k). The “0.2” used in equation 15 above may be a tunable factor and may be expressed as “C”. “C” may be the maximum standard level of trust to the noninvasive sensor's readings. “C” may vary, for example between about 0.05 to about 0.4. Accordingly, X(k) may be calculated as:

X(k)=C·max(0,min(1,(G_{CGM}(k)−G_{NI}(k))/(G_{CGM}(k−1)−G_{NI}(k−1)))) (Eq. 15a).

The value of the trust weight X(k) may be increased to a certain maximum value, such as up to 0.4, depending on the rate of change of the current glucose readings. Particularly, the higher the rate of change of the current glucose value, the less reliable are the readings provided by the invasive glucose sensors. Therefore, the trust weight on the noninvasive sensors may be increased. In one exemplary embodiment, this may be expressed as follows:

$${X}_{f} ( k ) = min ( 0.4 , max ( 0 , X ( k ) \cdot \frac{{G}_{C G M} ( k ) - {G}_{C G M} ( k - 1 )}{10} ) ) . \qquad ( Eq . 16 )$$

This formulation allows the trust index to increase up to 0.4, or 2× the maximum standard level of trust to the noninvasive sensor's readings, if the rate of change of current invasive glucose sensor glucose level readings exceed 20 mg/dL/5 minutes (i.e., 1 cycle). “20 mg/dL/5 minutes” may be a tunable factor and may also be referred to as “ROC_{Trust}, max” Accordingly, equation 16 may be expressed as:

$${X}_{f} ( k ) = min ( 2 \cdot C , max ( 0 , X ( k ) ) \cdot \frac{{G}_{CGM} ( k ) - {G}_{CGM} ( k - 1 )}{R O {C}_{Trust , max} / 2} ) ) . \qquad ( Eq . 16 a )$$

Alternately, in cases where the degradation in the reliability of the noninvasive glucose sensor may be greater than the invasive glucose sensor in cases of high rates of change, an inverted implementation of this value can be determined, as follows:

$${X}_{f} ( k ) = min ( 0.2 , X ( k ) \cdot \frac{10}{{G}_{CGM} ( k ) - {G}_{CGM} ( k - 1 )} ) ) . \qquad ( Eq . 17 )$$

This formulation allows the trust index to decrease asymptotically down to 0, with a 50% reduction to 0.1 if the rate of change in invasive glucose sensor's glucose readings reach 20 mg/dL/5 minutes. Equation 17 may also be expressed as:

$${X}_{f} ( k ) = min ( C , X ( k ) \cdot \frac{R O {C}_{Trust , max} / 2}{{G}_{CGM} ( k ) - {G}_{CGM} ( k - 1 )} ) \qquad ( Eq . 17 a )$$

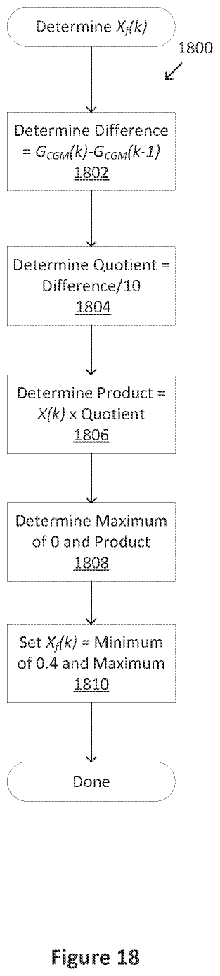

FIG. 18 depicts a flowchart 1800 of illustrative steps that may be performed in exemplary embodiments to determine X_{f}(k) for Eq. 16. At 1802, the difference between the glucose level reading of the invasive glucose sensor for the current cycle k, G_{CGM}(k), and the glucose reading of the invasive glucose sensor for the predecessor cycle k−1, G_{CGM}(k−1), is determined. At 1804, the difference is divided by 10 to yield a quotient. At 1806, a product of the quotient and X(k) is determined. At 1808, the maximum of the product and 0 is determined to ensure that a positive value is chosen. At 1810, X_{f}(k) is set as the minimum of 0.4 (i.e., the maximum trust weight permitted) and the previously determined maximum. Similar calculations can be made for Equation 17 in cases where the noninvasive glucose sensor is less reliable than the invasive glucose sensor under conditions of rapidly changing glucose.

The glucose level ROC data also may be used to confirm that delivery of insulin from the medicament delivery device 102 to an interstitial space under the skin of the user has occurred. The ability to identify that an intended insulin delivery did not occur may allow for quick identification of an issue with the medicament delivery device 102, such as, for example, a needle or cannula obstruction, a kink in the delivery path, unintended leakage of medicament, or the detector being unseated from the interstitial fluid. Being able to quickly identify such issues may prevent a hyperglycemic event.

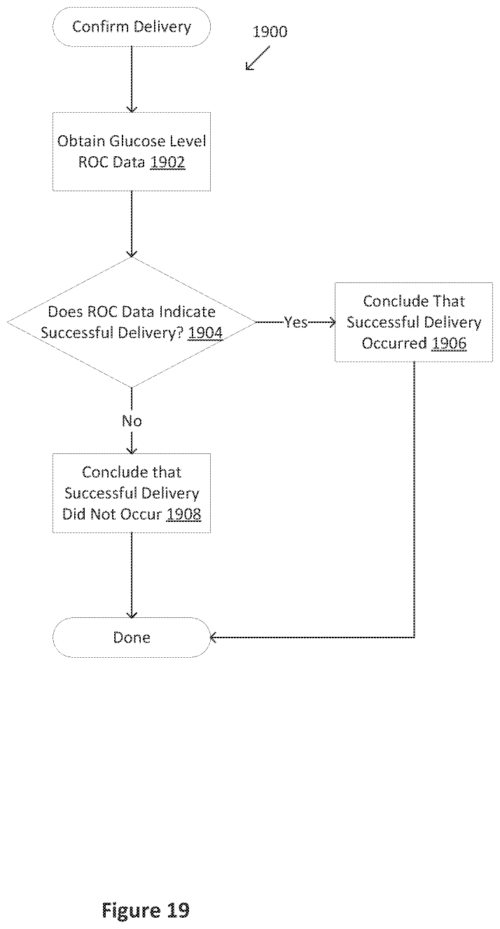

FIG. 19 depicts a flowchart 1900 of illustrative steps that may be performed in exemplary embodiments to confirm delivery of medicament, such as insulin, into the interstitial space of the user. At 1902, glucose level ROC data may be obtained from the non-invasive glucose sensor 152. The data may contain one ROC value for a given time or multiple ROC values for different times, such as over successive times, like over consecutive cycles. At 1904, a check is made of whether the glucose level ROC data indicates delivery of the medicament into the interstitial space of the user. This may entail, for example, comparing the glucose level ROC values to a threshold. One may expect that the ROC of the glucose level of the user is negative and of a certain magnitude if the medicament was successfully delivered. Alternatively, the trend of multiple glucose level ROC values may be examined to identify successful or unsuccessful delivery of the medicament into the interstitial space. One may expect the trend to indicate a negative ROC trend of non-negligible magnitude if the medicament was successfully delivered. As yet another alternative, the ROC of the glucose level ROC values may be compared to a threshold to determine whether the ROC indicates successful delivery of the medicament. At 1906, if the ROC data indicates successful delivery, a conclusion is reached that the medicament was successfully delivered. Conversely, if the ROC data indicates unsuccessful delivery, at 1908, a conclusion is reached that the medicament was not successfully delivered. Remedial action may be taken in response.

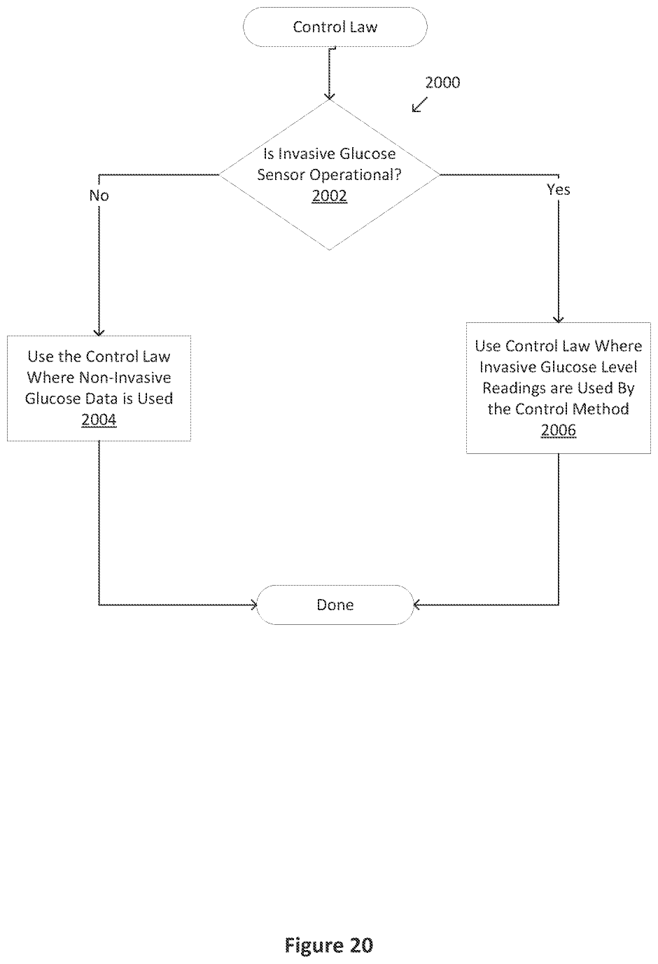

In some exemplary embodiments, the control application 116 or 120 may apply different control laws in determining and delivering medicament doses. A first control law may be applied when the glucose level values that are input to the control method originate from an invasive glucose sensor, like a CGM. In contrast, a second control law may be applied if the non-invasive glucose sensor provides the glucose sensor input to the control method. FIG. 20 depicts a flowchart 2000 of illustrative steps that may be performed in exemplary embodiments regarding the control laws. At 2002, a check may be made whether the invasive glucose sensor is operational or not, or, alternatively, whether glucose readings are being received from an invasive glucose sensor such as a CGM, or still further, whether glucose readings are being received from an invasive glucose sensor such as a CGM after a period of time has elapsed, such as a warm-up period of time after a CGM has been applied on the user's skin. Typically, invasive glucose sensors like CGMs have a limited lifetime (such as and need to be replaced every 7 to 14 days). While such CGMs are removed and a new CGM is being prepared, the invasive glucose sensor (e.g., the CGM) is not operational. It takes about 20 to 30 minutes for the replacement CGM to be initialized and fully operational. During the time window that the invasive glucose sensor is not operational, the glucose level of a user may change substantially. Hence, at 2004, if the invasive glucose sensor is not operational or readings are not being received from the invasive glucose sensor, a first control law is used where non-invasive glucose data is used in a manner such as described above. This helps to better regulate the glucose level of the user in such a circumstance. In contrast, if the invasive glucose sensor is still operational, at 2006, a second control law for using invasive glucose sensor readings is used. By way of example, a first control law that may be used while relying on sensor data from a non-invasive sensor may trust the data received to a lesser (or greater) extent than when a second control law is being used while relying on sensor data from an invasive sensor. For example, when a first control law is being used in the algorithm, the discrete glucose values that may be received from a non-invasive sensor may be trusted less than when a second control law is being used in the algorithm, i.e., when discrete glucose values are being received from an invasive sensor. Still further, under a first control law, an algorithm may rely on ROC data from the non-invasive sensor rather than discrete glucose sensor values from the non-invasive sensor; and under a second control law, an algorithm may rely on discrete glucose sensor values from an invasive sensor in addition to (or in some embodiments, instead of) ROC data from the invasive glucose sensor or the non-invasive glucose sensor.

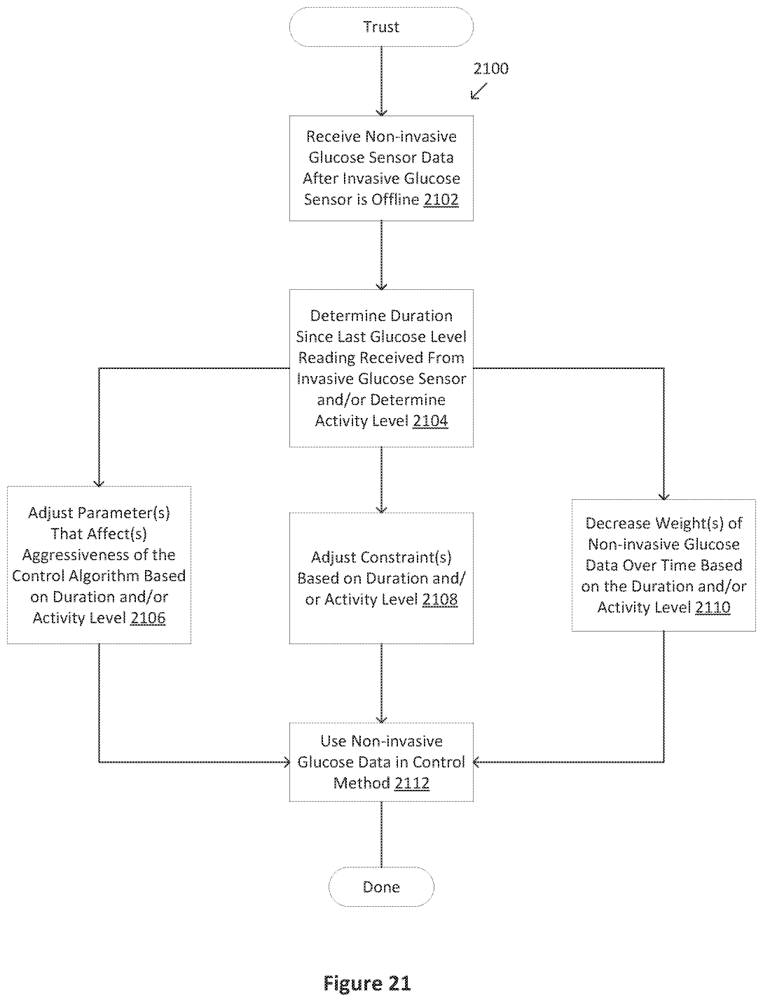

Non-invasive glucose sensor values may be trusted to varying degrees based on various factors. FIG. 21 depicts a flowchart of illustrative steps that may be performed regarding trust of non-invasive glucose sensor data based on factors. At 2102, non-invasive glucose sensor data is received after the invasive glucose sensor is offline. In this circumstance, a duration since the last glucose level reading from the invasive glucose sensor was received and/or an activity level of the user may be determined at 2104. In general, the trust level of the non-invasive glucose sensor data decreases over time as there is greater risk that the data diverges from the invasive glucose sensor data. The trust in the non-invasive glucose sensor data also decreases as the activity level of the user is elevated since the exercise may cause the glucose level of user to drop.

The control application 116 or 120 may then perform one or more of steps 2106, 2108, or 2110. At 2106, parameter(s) that affect(s) the aggressiveness of the control algorithm may be adjusted based on the determined duration and/or the activity level. For instance, a cost function or other algorithm may be adjusted to be less aggressive as the duration value becomes larger, such as by adjusting a coefficient for a glucose deviation cost in an exemplary cost function. With the decreased level of trust in the non-invasive glucose sensor data, the system wishes to be less aggressive to minimize the risk of the user becoming hypoglycemic. Similarly, as the activity level becomes elevated and persists, the non-invasive glucose sensor data is trusted less; hence, the parameter(s) may be adjusted to be more conservative. The magnitude of the adjustment and the magnitudes of the duration and the activity level may be determined empirically and may be customized for individual users.

At 2108, constraint(s) may be adjusted based on the duration and/or the activity level. As the trust decreases, the constraint(s) may be increased to avoid hypoglycemia. For instance, an increase of the constraint(s) may be that the maximum basal dose of medicament per cycle is decreased, and/or the total aggregate medicament delivered per a time period (like an hour) is decreased. At 2110, weight(s) may be assigned to the non-invasive glucose sensor data in predicting future glucose level values for the user. These weights may be diminished over time so that as the duration increases, the non-invasive glucose data has less weight. Similarly, the weight of the non-invasive glucose level values may be decreased as the activity level of the user increases. At 2112, the non-invasive glucose sensor data is used in the control method with one or more of the adjusted parameter(s), adjusted constraint(s), and decreased weight(s).

Software related implementations of the techniques described herein may include, but are not limited to, firmware, application specific software, or any other type of computer readable instructions that may be executed by one or more processors. Hardware related implementations of the techniques described herein may include, but are not limited to, integrated circuits (ICs), application specific ICs (ASICs), field programmable arrays (FPGAs), and/or programmable logic devices (PLDs). In some examples, the techniques described herein, and/or any system or constituent component described herein may be implemented with a processor executing computer readable instructions stored on one or more memory components.

In addition, or alternatively, while the examples may have been described with reference to a closed loop algorithmic implementation, variations of the disclosed examples may be implemented to enable open loop use. The open loop implementations allow for use of different modalities of delivery of insulin such as smart pen, syringe or the like. For example, the disclosed applications and algorithms may be operable to perform various functions related to open loop operations, such as the generation of prompts requesting the input of information such as weight or age. Similarly, a dosage amount of insulin may be received by the applications or algorithms from a user via a user interface. Other open-loop actions may also be implemented by adjusting user settings or the like in an application or algorithm.

Some examples of the disclosed device may be implemented, for example, using a storage medium, a computer-readable medium, or an article of manufacture which may store an instruction or a set of instructions that, if executed by a machine (i.e., processor or microcontroller), may cause the machine to perform a method and/or operation in accordance with examples of the disclosure. Such a machine may include, for example, any suitable processing platform, computing platform, computing device, processing device, computing system, processing system, computer, processor, or the like, and may be implemented using any suitable combination of hardware and/or software. The instructions may be executed by a processor. The instructions may also be performed by a plurality of processors for example in a distributed computer system. The computer-readable medium or article may include, for example, any suitable type of memory unit, memory, memory article, memory medium, storage device, storage article, storage medium and/or storage unit, for example, memory (including non-transitory memory), removable or non-removable media, erasable or non-erasable media, writeable or re-writeable media, digital or analog media, hard disk, floppy disk, Compact Disk Read Only Memory (CD-ROM), Compact Disk Recordable (CD-R), Compact Disk Rewriteable (CD-RW), optical disk, magnetic media, magneto-optical media, removable memory cards or disks, various types of Digital Versatile Disk (DVD), a tape, a cassette, or the like. The instructions may include any suitable type of code, such as source code, compiled code, interpreted code, executable code, static code, dynamic code, encrypted code, programming code, and the like, implemented using any suitable high-level, low-level, object-oriented, visual, compiled and/or interpreted programming language. The non-transitory computer readable medium embodied programming code may cause a processor when executing the programming code to perform functions, such as those described herein.

The foregoing description of examples has been presented for the purposes of illustration and description. It is not intended to be exhaustive or to limit the present disclosure to the precise forms disclosed. Many modifications and variations are possible in light of this disclosure. It is intended that the scope of the present disclosure be limited not by this detailed description, but rather by the claims appended hereto. Future filed applications claiming priority to this application may claim the disclosed subject matter in a different manner and may generally include any set of one or more limitations as variously disclosed or otherwise demonstrated herein. The present disclosure furthermore relates to computer programs comprising instructions (also referred to as computer programming instructions) to perform the aforementioned functionalities. The computer programs of the present disclosure may be for example preinstalled on, or downloaded to the medicament delivery device, management device, fluid delivery device, e.g. their storage.

What is claimed is:

1. An insulin delivery device for delivering insulin to a user, comprising:
a non-transitory computer-readable storage medium storing processor-executable instructions;
a processor for executing the processor-executable instructions to cause the processor to:
receive rate of change (ROC) data regarding an ROC of a glucose level of the user; and
perform at least one of the following:
use the ROC data without using glucose level data to determine basal insulin delivery dosages;
use the ROC data without using the glucose level data to determine a lowest glucose cost among candidate basal insulin delivery dosages;
use the ROC data without using the glucose level data to detect an imminent hypoglycemic event or an imminent hyperglycemic; or
use the ROC data without using glucose level data to confirm that insulin was delivered to the user.

2. The insulin delivery device of claim 1, where the processor uses the ROC data without using glucose level data to determine basal insulin delivery dosages, the processor-executable instructions cause the processor to analyze the ROC data to identify that a projected glucose level increase is projected.

3. The insulin delivery device of claim 2, wherein the processor-executable instructions further cause the processor to increase an insulin delivery rate by the insulin delivery device to the user to compensate for the projected glucose level increase.

4. The insulin delivery device of claim 2, wherein a magnitude of the increase of insulin delivery rate depends upon the ROC data and a target glucose level of the user.

5. The insulin delivery device of claim 1, where the processor uses the ROC data without using glucose level data to determine basal insulin delivery dosages, the processor-executable instructions cause the processor to analyze the ROC data to identify that a projected glucose level decrease is projected.

6. The insulin delivery device of claim 5, wherein the processor-executable instructions further cause the processor to decrease an insulin delivery rate by the insulin delivery device to the user to compensate for the projected glucose level decrease.

7. The insulin delivery device of claim 1, where the processor uses the ROC data without using the glucose level data to determine glucose cost of candidate basal insulin delivery dosages, the processor-executable instructions cause the processor to predict a ROC for a time period from the rates of change of preceding time periods.

8. The insulin delivery device of claim 7, wherein the processor-executable instructions further cause the processor to use a cost function that predicts costs of candidate insulin doses using the predicted ROC for the time period.

9. The insulin delivery device of claim 8, wherein the cost function includes a glucose cost component that is determined based on the predicted ROC for the time period.

10. The insulin delivery device of claim 8, wherein the processor-executable instructions further cause the processor to choose a selected one of the candidate insulin doses with a lowest cost as determined by the cost function and to cause the selected one of the insulin doses to be delivered to the user.

11. An insulin delivery device for delivering insulin to a user, comprising:
a non-transitory computer-readable storage medium storing processor-executable instructions;
a processor for executing the processor-executable instructions to cause the processor to:
receive rate of change (ROC) data from a noninvasive sensor, the ROC data indicating an ROC of glucose concentration for the user;
modify a weight coefficient of a glucose cost component of a cost function based on the ROC data;
select an insulin dose among candidate insulin doses to be delivered to the user in an operational cycle of the drug delivery device using the cost function, wherein the selected dose has a better cost relative to others of the candidate insulin doses; and
cause the selected insulin dose to be delivered to the user during the operational cycle.

12. The insulin delivery device of claim 11, wherein the cost function includes a glucose cost component and an insulin cost component, wherein the glucose cost component is based on how much predicted glucose concentrations of the user will vary from target values if a given insulin dose is delivered to the user in a current operational cycle of the drug delivery device and wherein a weight coefficient of the glucose cost component is calculated using the ROC data.

13. The insulin delivery device of claim 12, wherein the weight coefficient of the glucose cost component increases a magnitude of the glucose cost component if the ROC data indicates that the glucose concentration is increasing and if a difference between the glucose concentration of the user for the current operational cycle and a glucose concentration of the user for a predecessor operational cycle is positive, or if the glucose concentration is decreasing and if a difference between the glucose concentration of the user for the current operational cycle and a glucose concentration of the user for a predecessor operational cycle is negative.

14. The insulin delivery device of claim 12, wherein the weight coefficient of the glucose cost component decreases a magnitude of the glucose cost component if the ROC data indicates that the glucose concentration is increasing and if a difference between the glucose concentration of the user for the current operational cycle and a glucose concentration of the user for a predecessor operational cycle is negative, or if the glucose concentration is decreasing and if a difference between the glucose concentration of the user for the current operational cycle and a glucose concentration of the user for a predecessor operational cycle is positive.

15. An insulin delivery device for delivering insulin to a user, comprising:
a non-transitory computer-readable storage medium storing processor-executable instructions;
a processor for executing the processor-executable instructions to cause the processor to:
receive a rate of change (ROC) reading for an operational cycle of the insulin delivery device from a noninvasive glucose sensor;
determine an offset between the ROC reading and a corresponding subcutaneous glucose sensor reading for the operational cycle;
determine a calibrated subcutaneous glucose sensor reading for the operational cycle using the offset; and
use the calibrated subcutaneous glucose sensor reading to determine an insulin dose for the operational cycle; and
cause the insulin dose to be delivered by the insulin delivery device to the user during the operational cycle.

16. The insulin delivery device of claim 15, wherein the processor-executable instructions further cause the processor to calculate an estimate of an ROC of the subcutaneous glucose sensor readings using the ROC reading from the noninvasive sensor for a current operational cycle and an ROC reading from the noninvasive sensor for a predecessor operational cycle.

17. The insulin delivery device of claim 16, wherein the processor-executable instructions further cause the processor to determine a value equal to a ratio of a difference between the subcutaneous glucose sensor reading for the current operational cycle and a subcutaneous glucose sensor reading for the predecessor operational cycle and the estimate of an ROC of the subcutaneous glucose sensor readings.

18. The insulin delivery device of claim 17, wherein the offset is determined as a difference between the subcutaneous glucose sensor reading for the current operational cycle and a product of ratio and the ROC reading from the noninvasive sensor for the current operational cycle.

19. The insulin delivery device of claim 18, wherein the calibrated subcutaneous glucose sensor reading for the operational cycle is determined by adding the offset to the product of ratio and the ROC reading from the noninvasive sensor for the current operational cycle.

20. The insulin delivery device of claim 15, wherein the subcutaneous glucose sensor is a continuous glucose monitor.
